(19) United States
(12) Patent Application Publication
(10) Pub. No.: US 2025/0032753 A1
(43) Pub. Date: Jan. 30, 2025

(54) ROBOTIC CATHETER WITH DISTAL GUIDE RING WITH NOTCH

(71) Applicant: Canon U.S.A., Inc., Melville, NY (US)

(72) Inventors: Benedict Shia, Needham, MA (US); Anne Yujin Edge, Somerville, MA (US); Takahisa Kato, Brookline, MA (US)

(21) Appl. No.: 18/781,444

(22) Filed: Jul. 23, 2024

Related U.S. Application Data
(60) Provisional application No. 63/515,315, filed on Jul. 24, 2023.

(51) Int. Cl. A61M 25/01; A61B 1/005

(52) U.S. Cl. CPC A61M 25/0147; A61B 1/0057

(57) ABSTRACT

A steerable medical instrument, such as a catheter or endoscope, comprises a tubular body having non-steerable section and steerable sections arranged from a proximal end to a distal end thereof. The tubular body defines a tool channel and a plurality of wire conduits formed along the wall of the tubular body. The steerable section includes wire-guiding members arranged in lengthwise direction alternated with void regions.

### CROSS REFERENCE TO RELATED PATENT APPLICATIONS

This application claims priority from U.S. Provisional Patent Application No. 63/515,315, filed on Jul. 24, 2023, in the United States Patent and Trademark Office, the disclosure of which is incorporated by reference herein, in its entirety.

### FIELD OF THE DISCLOSURE

The present disclosure generally relates to steerable medical devices. More particularly the present disclosure exemplifies various embodiments of a steerable medical device having cable strain relief elements. The steerable medical device is applicable to interventional medical tools and instruments, such as endoscopes and catheters, configured to navigate through intraluminal tortuous paths under manual and/or robotic control.

### BACKGROUND OF THE DISCLOSURE

Bendable medical instruments such as endoscopes and catheters generally include an elongated flexible tubular shaft commonly referred to as a sleeve or sheath guide, which has a cylindrical opening extending from a proximal end to a distal end. One or more tool channels extend along (typically inside) the cylindrical opening to allow access to end effectors located at the distal end of the sheath. In addition to end effectors, endoscopes and catheters typically include imaging, illumination, and sensing components at the distal end of the flexible shaft to enable safe navigation through non-linear lumens or tortuous pathways within the body of a patient. This type of medical instrument is configured to provide flexible access to target areas while retaining torsional and longitudinal rigidity so that physicians can control the end effectors located and imaging devices at the distal end by maneuvering dial wheels, control knobs, or joystick controllers a proximal end of the instrument.

Steerable medical instruments transfer motions of the hands of a user (such as an endoscopist) from a handle of the instrument to the distal end of the sheath using one or more long and flexible cables, which are attached to actuating elements on the handle side and to one or more joint mechanisms at the distal end of the sheath. This is similar to transferring motions from a muscle of a human body to a joint through a tendon. Accordingly, in steerable medical instruments, actuation of the joint mechanisms by the cable is also referred to as tendon-driven actuation. To improve accuracy of tendon-driven actuation, ensure safety and minimize patient trauma or discomfort, robotically controlled actuation and image guided operation of steerable instrument is preferred.

There is a wide variety of robots applicable to steerable medical devices, and all robots use a mechanical structure of several links and actuators to operate the cables and joints with one or more degrees of freedom of motion. The actuators driving the tendon cables may include a variety of different types of mechanisms capable of applying a force to a tendon, e.g., electromechanical motors, pneumatic and hydraulic cylinders, pneumatic and hydraulic motors, solenoids, shape memory alloy (SMA) wires, electronic rotary actuators or other devices or methods as known in the art. An example of such robotic controlled instrument is the Da Vinci Surgical System made by Intuitive Surgical Inc.

Frequently, robotically controlled intraluminal steerable instruments, such as catheters and endoscopes, incorporate a number of different electronic components. These components typically have electrical wiring which gets routed internally through the wall of the tubular shaft. The electrical wiring cables can be routed such that they are offset from the instrument's central axis. In such a design, when the steerable instrument takes on a curved geometry, the length of wire required to adapt to that geometry becomes longer than the original wire length. This creates a strain condition on the electrical cable which in turn can negatively impact catheter maneuverability and may damage the electrical cable itself.

The current state of the art can be seen in United States Patent Application number 2021/0259790 which discloses a robotic catheter having an electromagnetic sensor 190 on a tip. The electromagnetic sensor comprises a coil with a straight shape. The distal tip portion of the catheter encompasses the electromagnetic sensor and maintains the straight shape of the electromagnetic sensor. As shown in FIGS. 1A and 1B, the distal guide ring is also attached to the distal tip portion and forms the rigid distal portion with the distal tip portion. The distal guide ring anchors drive wires (not shown in the figure) and includes a notch 512. The notch lets an electrical cable 112 of the electromagnetic sensor go through to the proximal side of the robotic catheter to connect to the controller.

However, the shortcomings of the longer rigid distal portion make catheter manipulation more challenging, especially in maneuvering into confined bifurcations of the lungs and other tortious pathways, and to perform dexterous tip motion in the confined space because the rigid distal portion with the distal tip portion and the distal guide ring cannot be bent.

Therefore, there is a need for an improved steerable medical instrument having improved flexibility and an abbreviated distal tip/portion for efficiently traveling through tortuous paths without causing pain or discomfort to a patient and without causing excessive strain in the delicate electrical wiring embedded within the tubular shaft of the steerable instrument.

### SUMMARY

Thus, to address such exemplary needs in the industry, the presently disclosed apparatus teaches a robotically steerable medical instrument, such as an endoscope or a catheter, comprising a catheter body having a proximal section configured to be mechanically coupled to an actuator, and a distal section configured to be actuated by the actuator, the catheter body having a tool channel which extends along a central axis from a proximal end to a distal end of the catheter body, and having at least one control wire and at least one electrical cable arranged lengthwise along a wall of the catheter body, the catheter body further comprising a distal tip configured at the distal end of the distal section, the distal tip configured to house at least a portion of an electrical component, wherein the distal section includes a plurality of wire-guiding members arranged at a predetermined distance from each other so as to form a plurality of void regions alternately interposed with the plurality of wire-guiding members in the lengthwise direction of the catheter body, and a distal guide ring configured at the distal end of the distal section for attaching the at least one control wire, wherein the at least one control wire links the distal guide ring to the actuator and is configured to transfer an actuating force from the actuator to the distal guide ring of the catheter body so as to bend at least a portion of the distal section, wherein the electrical cable is configured to establish an electrical connection between an electrical circuit located outside the catheter body and the electronic component arranged within at least a portion of the distal tip, and wherein the electrical component bridges the distal tip and distal guide ring.

In another embodiment of the subject innovation, the electrical component is selected from the groups consisting of an endoscope camera, a position sensor, an orientation sensor, or combinations thereof.

In a further embodiment, the distal tip comprises a cap to protect the distal tip. In a further embodiment, the distal tip comprises a first notch for housing at least a portion of the electrical component.

It is further contemplated herein that each of the wire-guiding members comprises a second notch for passage of the electrical cable through the distal section.

In an additional embodiment, the electrical cable includes strain relief elements arranged in one or more void regions such that when the electrical cable is subjected to a tensile load by the actuating force bending the distal section of the catheter body, the strain relief elements within the one or more void regions minimize the tensile load on the electrical cable.

In yet another embodiment of the subject innovation, the strain relief elements include first portions of the electrical cable which are loosely arranged within the one or more void regions such that, within the one or more void regions, the first portions of the electrical cable can freely move, slide, and/or tighten in response to the actuating force bending the distal section to thereby provide strain relief to, or minimize the tensile load of, the electrical cable.

It is also contemplated that the subject steerable medical instrument further comprises an inner sheath configured to enclose at least part of the tool channel, wherein the one or more wire-guiding members and the void regions interposed with the one or more wire-guiding members are arranged around the inner sheath, wherein both the electrical cable and the control wires surround the inner sheath, wherein the one or more wire-guiding members are attached to the inner sheath by reflowing material of the wire-guiding members and/or material of the inner sheath onto each other.

In another embodiment, the steerable medical instrument further comprises an outer sheath configured to enclose at least part of the distal section of the catheter body, wherein the outer sheath encloses the one or more wire-guiding members and the void regions interposed with the one or more wire-guiding members, wherein the outer sheath surrounds both the electrical cable and the control wires within the void regions interposed with the one or more wire-guiding members, and wherein, when the tensile load by the actuating force bends the distal section of the catheter body, the outer sheath is configured to prevent prolapse of the electrical cable and/or control wires within the void regions interposed with the one or more wire-guiding members.

In yet another embodiment, the electrical cable is made of one or more strands of electrically conducing wire, wherein at least part of the one or more strands of electrical conducting wire are wound into a twisted pair cable or weaved into a braided multi-strand cable.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objectives, features and advantages of the present disclosure will become apparent from the following description when taken in conjunction with the accompanying figures showing illustrative embodiments of the present disclosure.

FIGS. 1A and 1B provide side and front views, respectively, of the existing art in the field.

FIG. 2A illustrates a general structure of a continuum robot system configured to control a steerable instrument, according to one embodiment of the present disclosure. FIG. 2B illustrates in more detail the steerable instrument 100 having an elongate flexible sheath (elongate tubular body) with at least one tool channel and a plurality of wire conduits 104 extending along the length of the sheath, according to one or more embodiment of the subject innovation.

FIG. 3A is a perspective view of an exemplary steerable instrument having a non-steerable section and a steerable section, in a non-bent state. FIG. 3B shows an example of bending or steering a single segment of steerable section of steerable instrument, according to one or more embodiment of the subject innovation.

FIG. 4 illustrates a close-up view of the steerable section located at the distal end of the steerable instrument, according to one or more embodiment of the subject innovation.

FIG. 5 is an image of a cross-sectional section of the steerable structure on the A-A line found in FIG. 4, according to one or more embodiment of the subject innovation.

FIG. 6 provides a side view of the steerable section located at the distal end of the steerable instrument, according to one or more embodiment of the subject innovation.

FIG. 7 illustrates a close-up view of the steerable section located at the distal end of the steerable instrument, according to one or more embodiment of the subject innovation.

FIG. 8 illustrates a close-up view of the steerable section located at the distal end of the steerable instrument, according to one or more embodiment of the subject innovation.

FIG. 9 provides a side view of the steerable section located at the distal end of the steerable instrument, according to one or more embodiment of the subject innovation.

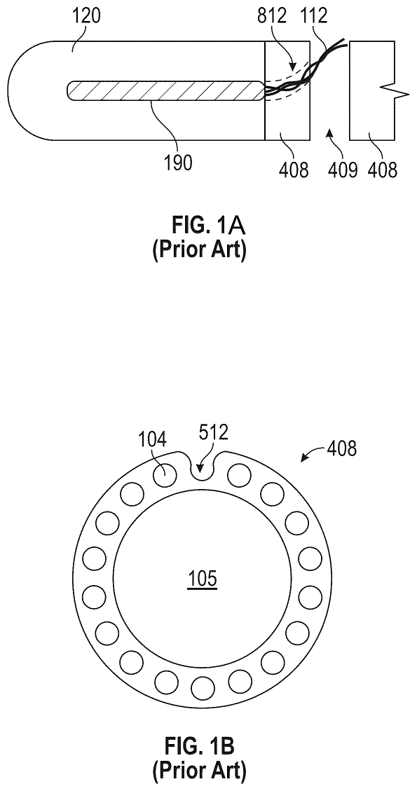

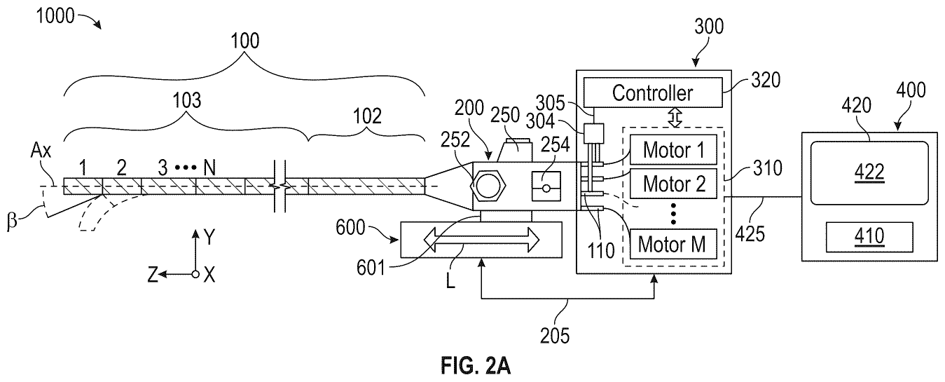

### DETAILED DESCRIPTION OF DISCLOSURE

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope and spirit of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive, exclusive, or otherwise limit or restrict the claims to the precise forms and configurations shown in the drawings and disclosed in the following detailed description.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The present disclosure generally relates to medical devices and exemplifies embodiments of robotically controlled steerable instrument which may be applicable to an endoscope (e.g., a bronchoscope), or a catheter for optical coherence tomographic (OCT) or intravascular ultrasound (IVUS), or a combination of such apparatuses (e.g., a multi-modality optical probe). The embodiments of the steerable instrument and portions thereof are described in terms of their positon/orientation in a three-dimensional space. As used herein, the term “position” refers to the location of an object or a portion of an object in the three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to a total six degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, when applicable, the terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

Specific embodiments of the present disclosure are directed to improved robotically controllable endoscopes or catheters for application in minimally invasive surgery (MIS) procedures. MIS procedures involve the use of long rigid or flexible surgical instruments that are inserted into the body of a patient through small incisions or natural orifices. Today, there is wide range of well-known endoscopic procedures. An important aspect of MIS endoscopy is the ability to “see” inside the body of the patient by directly inserting an imaging device into the area of interest. As the imaging device, most endoscopes use a high-resolution camera and a light source at the endoscope tip. The endoscope tip can be actively steered either manually by two thumb-controlled dials, or by a robotic actuator at the proximal end. Insertion and retraction of the endoscope into the patient body can also be performed either manually or robotically. Throughout this disclosure, working principles and novel improvements for robotic controlled endoscopic devices are described in detail. The application of such endoscopic devices includes procedures for both diagnostic and therapeutic purposes.

### <Configuration and Operation of a Steerable Medical Instrument>

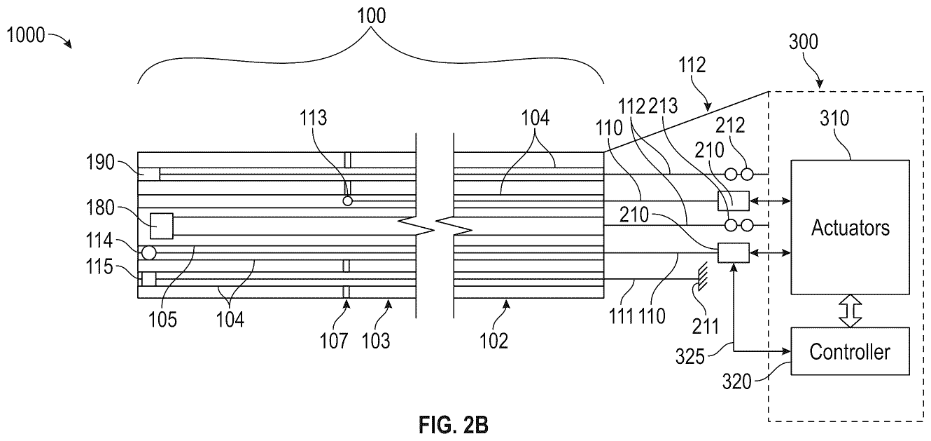

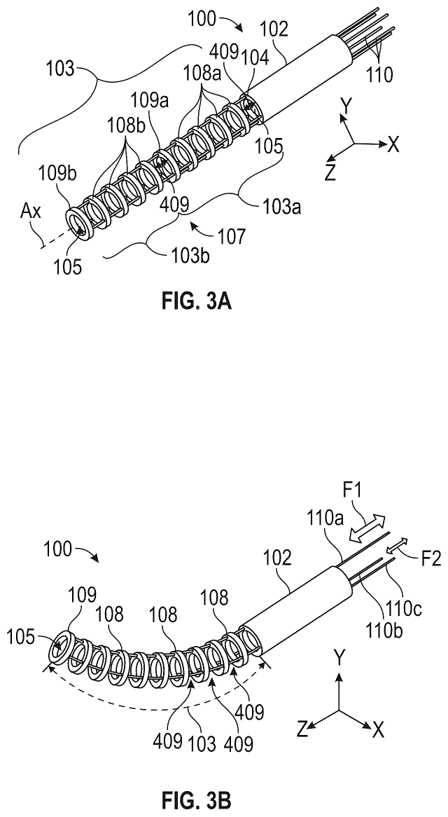

A general configuration and operation principles of steerable instrument 100 controlled by a robot system 1000 is described with respect to FIGS. 2A, 2B, 3A and 3B. As used herein, the steerable instrument 100 refers to a steerable catheter or endoscope having at least one steerable section which is manipulated by a mechanism which may be driven manually by operators or robotically by actuators. The robot system 1000 can include a continuum or multi-segment robot configured to take continuously curved geometries to move the endoscope probe or catheter longitudinally along tortuous paths. An example of a continuum robot is a snake-like endoscopic device, as described and presently embodied in applicant's previous patent or patent application publications including U.S. Pat. No. 9,144,370, patent publications US 2015/0088161; US 2018/0192854; US 2018/0243900; US 2018/0311006; US 2019/0015978; and US 2019/0105468, as well as in international publications WO2018204202; WO/2020/086749; WO/2020/092096; and WO/2020/092097, each of which are hereby incorporated by reference in their entirety for all purposes.

FIG. 2A illustrates a general structure of a continuum robot system 1000, according to one embodiment of the present disclosure. The system 1000 includes a computer system 400 (e.g. a system console), a robotic control system 300, and a steerable instrument 100 which is connected to the control system 300 via a handle 200. The steerable instrument 100 has a generally tubular shape with a proximal non-steerable section 102 and a distal steerable section 103 arranged along a central longitudinal axis Ax. The distal steerable section 103 includes a plurality of bending segments (1, 2, 3 . . . . N); and at least one of these bending segments can be mechanically actuated by one or more control wires to bend the steerable instrument 100 at an angle β with respect to the longitudinal axis.

The steerable instrument 100 is a medial-grade steerable shaft having dimensions appropriate to reach a patient's organ depending on the specific application. The steerable and non-steerable sections of instrument 100 form an elongated tubular body coupled to, and controlled with/by, the handle 200. Expressed in the X-Y-Z Cartesian coordinate system, the tubular body of the steerable instrument 100 has a tubular cross section in an X-Y plane, and the longitudinal axis Ax along the Z-axis perpendicular to the X-Y plane. In other words, the distal end of the steerable instrument 100 points towards the Z-direction, and is configured and dimensioned for insertion into a patient's body part either through a small surgical incision or a natural orifice.

The control system 300 generally includes a controller 320 and an actuator system 310. The controller 320 may include a proportional-integral-derivative (PID) controller or other digital signal processor (DSP) along with suitable software, firmware and peripheral hardware, as it is known to persons having ordinary skill in the art. PID or DSP-based controllers are generally dedicated integrated circuits; however DSP functionality can also be implemented by other circuits, for example, by using field-programmable gate array chips (FPGAs). Therefore, in some embodiments, the control system 300 can be connected to a computer system 400 via a network connection 425. The controller or computer system 400, along with suitable software, firmware and peripheral hardware, operated by a microprocessor or central processing unit (CPU) 410 controls the functions of the continuum robot system 1000, as described in the remainder of this disclosure. Among other functions, the computer system 400 can provide a surgeon or other user with an image display device 420, such as an LCD or OLED, configured as a monitor screen 422 configured to display images and a graphical user interface (GUI) with a touchscreen to interact and remotely operate the steerable instrument 100. Alternatively or in addition thereto, the control system 300 and/or handle 200 can be connected to a handheld controller, such as a gamepad controller or a portable controller device like a smartphone or tablet (not shown).

The actuator system 310 includes a plurality of actuators or actuating motors (Motor 1 through M) equal to a plurality of control wires 110 (also referred to as drive wires) necessary for actuating and steering the instrument 100. The robotic control system 300 also includes and/or controls one or more sensors 304. Sensors 304 can include a strain sensor and/or a position sensor for each control wire 110. These sensors 304 serve to detect and/or measure compressive and/or tensile forces applied by the actuators to drive each control wire 110. The sensors 304 also output a signal 305 corresponding to the amount of compressive and/or tensile force (an amount of strain) being applied to a control wire 110. The sensors 304 could also output a signal 305 corresponding to an amount of movement (a distance) of displacement for each actuated control wire 110, at any given point in time during a procedure. The output signals 305 from the sensors 304 (strain sensor and/or position sensor) for each control wire 110 are fed back to the controller 320 to control each actuator and control wire 110 individually with a feedback control loop. In this manner, each control wire 110 can be actively controlled to implement appropriate shaft guidance for navigating the instrument 100 through intraluminal paths of a patient's anatomy. During catheter navigation, the system continuously monitors the contact force that is exerted by the catheter tip by using a specially designed algorithm (e.g., as described in US 2007/0135803) and the sensors 304. If the contact force exceeds a preset limit, the system provides a warning, and catheter advancement is interrupted and the navigation path is corrected.

The handle 200 includes mechanical, electronic, electrical, and optical components which serve to provide electromechanical interconnection between the steerable instrument 100 and the control system 300. For example, the handle 200 may provide mechanical, electrical, and/or optical connections, and a data/digital acquisition (DAQ) system for interfacing the steerable instrument 100 with the control system 300. The handle 200 may also provide an access port 250, one or more mechanical dials or knobs 252, and a user interface 254. The one or more control wheels or knobs 252 are used to manually bend individual segments of the steerable section 103 in one or more directions. The access port 250 is used for insertion and extraction of tools into the tool channel 105, such as small forceps, needles, or electrocautery instruments and the like. The handle 200 is attachable to a robotic support platform 600 (e.g., a linear stage 601) to move the steerable instrument 100 in a linear direction L. The controller system 300 sends control signals to the support platform 600 and/or linear stage 601 via the handle 200 or/or an additional connection 205 such as a cable bundle.

As part of the user interface 254, the handle 200 may include one or more than one light emitting diode (LED) for providing operational status of the robotic steerable instrument 100 to a user. In an embodiment, the LED may include, for example, different light colors for respectively indicating normal operations (green light) and abnormal operations (red light). Alternatively, the LED may include blinking codes, for example, to indicate a type of abnormal operation. In addition, the user interface 254 may include an emergency on/off switch to manually stop actuation of the steerable instrument 100, in the event of an emergency.

FIG. 2B illustrates in more detail relevant parts of the steerable instrument 100. The steerable instrument 100 has an elongate tubular shaft (elongate body) also referred to as a sleeve or sheath guide. Along the shaft's length, there are one or more working channels 105 extending along (typically inside) the tubular shaft, and a plurality of wire conduits 104 extending along (typically within) the wall of the tubular shaft. The one or more tool channels 105 will be referred to as a “tool channel” for simplicity. The tool channel allows access for the tools (end effectors) to be delivered from an access port 250 to the distal end of the steerable section 103. The one or more channels 105 may also be used for sending or retrieving liquid or gaseous substances (e.g., air or water) to a target area, or for passing optical fibers and/or electric wires. Furthermore, the one or more channels 105 may be used for inserting one or more of a medical imaging device 180, such as an endoscope camera or a fiber-based imagining probe. An example of an endoscope camera includes, but is not limited to, a chip-on-tip (COT) camera, such as a camera with a miniature CMOS sensor and an illuminator arranged at the tip of the endoscope. Examples of fiber-based imaging probes include, but are not limited to, a near infrared auto-fluorescence (NIRAF) imaging probe, a spectrally encoded endoscopy (SEE) probe, an intravascular ultrasound (IVUS) probe, or an optical coherence tomography (OCT) imaging probe.

The steerable instrument 100 is configured to provide flexible access to intraluminal target areas with one or more than one bending curves to reach an intended target area usually located near (at a working distance from) the distal end of the instrument. Desirably, the steerable instrument 100 is capable of retaining torsional and longitudinal rigidity so that a user can control end effectors and/or imaging devices located at the distal end of the steerable section by remotely maneuvering the distal end of instrument 100 from the control knobs 252, the control system 300 and/or computer system 400. In order to provide such steerable functionality, the steerable instrument 100 is actuated with a plurality of control wires 110 which are arranged inside the wire conduits 104 along (typically within) the wall of the tubular shaft. Some of the control wires 110 are anchored at the distal end of the tubular shaft using wire anchors 114, and other control wires 110 can be anchored at certain predetermined distances from the distal end using wire anchors 113. In some embodiments, the steerable instrument 100 may include one or more support wires 111 (tendon wires). Support or tendon wires 111 can be optional at some locations of the steerable section, and are typically anchored at the distal end of the shaft with wire anchors 115, and can be mechanically grounded (attached) and/or biased to a support section 211 (e.g., the chassis, a mechanical spring, etc.,) of the endoscope handle 200.

In one exemplary embodiment, the steerable instrument 100 with six control wires 110 may have two pairs of control wires 110 (i.e., four control wires) anchored by wire anchors 113 in the midsection of the shaft (e.g., at one or more inflection points 107), and another pair of control wires 110 (two control wires) could be anchored by wire anchors 114 at the distal end of the shaft. In this manner, the steerable instrument 100 can have at least two (i.e., two or more) steerable sections controlled by 3 pairs of antagonistic control wires 110, where each wire extends through a separate wire conduit 104. According to one embodiment, the steerable instrument 100 has 3 locations with anchored control wires 110, and two locations with anchored support wires 111. The most distal anchor point has 3 control wires 110 and 3 support wires 111. The “middle” anchor point has 3 control wires and no support wires. And the proximal anchor point has 3 control wires and 3 support wires.

The wire conduits 104 allow anchorage and/or passage of control wires 110 used for steering (bending or twisting) at least one segment or section of the shaft. In addition, at least some wire conduits 104 can be used to pass one or more electrical cables 112. Electrical cables 112 are configured to establish an electrical connection between an electronic device arranged at the distal end or within the steerable section 103 and a terminal (212 or 213) or a signal processing circuit located outside of the proximal end of the instrument. For example, an electrical cable 112 can be used to connect one or more electromagnetic (EM) sensors 190 to first electrical terminals 212 located at the handle 200. In some embodiments, the wire conduits 104 can also be used to pass additional electrical cables 112 which can connect the imaging device 180 (a camera) to second electrical terminals 213 also located at the handle 200.

As used herein, an electrical cable 112 refers to a conductive cable or cable bundle including one or more wires configured to conduct an electrical signal (analog or digital) for connection of an electronic component, such an EM sensor or an endoscope camera (videoscope) located in or at the distal end of the catheter body, to signal processing circuitry located outside of the catheter body. An electrical cable 112 may be formed from one or more strands of an electrically conductive metal, such as silver, plated copper, copper, silver, gold, aluminum, and alloys thereof. The electrical cable 112 may be covered by a conventional electrically insulating material (jacket), such as polyurethane, polyester, nylon, and the like. An overall diameter of the electrical cable may be in a range of about 0.002 to 0.050 inches, or in a range of about 0.002 to 0.030 inches. These are merely examples of possible dimensions to illustrate the delicate nature of electrical cables 112 which are incorporated into steerable instrument 100. Since all aspects of these instruments continue to be miniaturized, it is expected that electrical cables 112 will also become smaller (thinner) and more delicate. Therefore, while an electrical cable measuring around 0.006 inch in diameter may be used, the cable may not have a round or circular cross section, but it may be more like oval cross section. For example, the electrical cable 112 could measure about 0.002×0.010 inches in cross sectional dimensions whether oval or rectangular. Therefore, a unique cable configuration that may have a non-round (non-circular) cross section may be advantageous in some situations. Moreover, depending on the specific application, the electrical cable 112 can be a coaxial cable, a twisted multi-cable bundle, or an irregularly wound or reinforced cable.

At the proximal end of the instrument 100, the handle 200 is configured to provide a mechanical linkage and an electromechanical interface between the steerable instrument 100 and the control system 300. In one embodiment, the handle 200 provides a plurality of electromechanical connections 210 (one connection for each of the control wires 110) so that an actuator system 310 can mechanically actuate each control wire 110. Each electromechanical connection 210 is in operative connection with the one or more sensors 304 to create a feedback loop 325 (based on signal 305) for controller 320. The controller 320 is used to electronically control the operation (movement) of each control wire 110 based on one or more of tensional, compressive, and/or torsional forces applied to each control wire 110.

FIGS. 3A and 3B each shows a perspective view of the steerable instrument 100 in a non-bended and bended state, respectively. The proximal non-steerable section 102 is a tubular (hollow) shaft with a central lumen which serves a tool channel 105 and a plurality of wire conduits 104 within the wall of the tubular shaft. The non-steerable section 102 of the steerable instrument has a hollow cylindrical shape with the longitudinal axis Ax extending along the Z-axis direction and a plurality of conduits or through holes extending within the wall of the cylindrical shape. In the non-bended state, the wire conduits 104 are preferably substantially equidistant, symmetrically arranged, and parallel to the longitudinal axis Ax. The steerable section 103 is divided into a plurality of bending segments 103a-103b joined at an inflection point 107 by a wire-anchor member 109a. Each bending segment of steerable section 103 includes a plurality of wire-guide members 108 (wire-guiding members or guide rings), and a wire-anchor member 109 (anchor member). Wire-guide members and wire-anchor members have the same shape and structure, but different function. In addition, some of the anchor members also act as wire-guiding members (or wire guides). For example, wire-anchor member 109a has control wires anchored to it and control wires passing through it non-anchored. A wire-anchor member 109b has control wires anchored to it, and it may also have support wires and/or an EM sensor attached to it.

In FIG. 3A, a first bending segment 103a includes four wire-guide members 108a and one wire-anchor member 109a. A second bending segment 103b includes four wire-guide members 108b and one wire-anchor member 109b. The wire-guiding members are arranged at predetermined distances from each other so as to form a plurality of void regions 409 along the steerable section 103. A plurality of control wires 110 extend from the proximal end to the distal end of the steerable instrument 100. The control wires 110 are initially passed through wire conduits 104 formed along (within) the wall of non-steerable section 102, and then routed through holes in the plurality of wire-guide members 108 and wire-anchor members 109. The anchor members 109a, 109b and guide members 108a, 108b have an annular shape (disk or ring shape) with the center axis thereof extending along the Z-axis direction, and the inner and outer diameter orthogonal to the axis direction. In the wire-guiding members, the wire conduits 104 (wire lumens) are through holes parallel to the axis direction formed around the surface of each disk. Here, those skilled in the art will understand that the proximal section 102 and wire-guiding members and wire-anchor members of distal section 103 are not limited to cylindrical-, disk- or ring-shaped structures. As long as the functionality of the steerable instrument 100 is met, the catheter body substructures can have any geometrical shape. For example, instead of disk, ring or cylindrical-shaped surfaces, at least one surface (e.g., the inner surface or outer surface or both) of the tubular shaft can have a hexagonal, octagonal, or similar polygonal shapes, with an overall dimension that fits within an inner diameter (ID) or outer diameter (OD) specified herein.

In FIG. 3A, among the plurality of control wires 110, some control wires pass through wire conduits 104 from the proximal end of the steerable instrument 100 (catheter body) to the first anchor member 109a, and some control wires 110 advance from the proximal end to the second anchor member 109b. The control wires 110 are fixedly attached to the anchor members, for example, by bonding, pinning, ultrasonic or heat or laser welding, pressure fitting, or attached by screws. All control wires 110 are coupled, at the proximal end thereof, to individual motors or actuators (i.e., to an actuator system 310, as shown in FIG. 2B).

The non-steerable section 102 has a function of transmitting an actuating force from the actuator system 310 to the bending segments of the steerable section 103 substantially without slack. To that end, the control wires 110 passing through the wire conduits 104 are driven in a linear push or pull direction L (parallel to the Z-axis direction), preferably without any buckling or slack. The control wires 110 can be metal wires, for example, piano-type wires, stainless-steel wires, nickel-titanium-alloy (nitinol) wires, or shape-memory-alloy (SMA) wires configured to push or pull a segment of the steerable section 103 according to an actuating force (tensile or compressive force) provided by one or more actuators of the control system 300. On the other hand, support wires 111 are non-actuated wires, but are configured to restore the steerable instrument to its passive position.

The steering motion (bending or twisting) of the steerable instrument 100 is explained next. For simplicity, the actuating of a single bending segment of the steerable section 103 is explained with reference to FIG. 3B. In the case of FIG. 3B, an exemplary steerable instrument 100 includes, from the distal end to the proximal end thereof, the steerable section 103 made of single bending segment and the non-steerable section 102 made of a tubular shaft with a central opening therein. A plurality of control wires 110a, 110b, and 110c extend from the proximal end to the distal end of the steerable instrument 100 along wire conduits 104 formed in (within) the wall of the non-steerable section 102 and through wire conduits 104 through holes) of guide members 108. One or more of the control wires 110 are fixedly coupled, at the distal end of the shaft, to an anchor member 109. The control wires 110 are also coupled, at the proximal end of the shaft, to individual actuators or motors of the actuator system 310. In this manner, the control wires 110 are selectively actuated by sliding with respect to the guide members 108 by the action of an actuator or motor connected at the proximal end of each control wire (refer to FIGS. 2A-2B). One of the three control wires 110 (e.g., control wire 110b in FIG. 3B) can fixed (or mechanically grounded) with respect to all guide members 108, and the remaining two control wires 110 (e.g., control wires 110a and 110c) are slideable with respect to the guide holes of the guide members 108.

In bending the steerable instrument 100, each control wire 110 is individually controlled by a respective actuator or motor. For example, in FIG. 3B, while control wire 110b may be fixed or anchored to anchor member 109, control wire 110a is pulled with a first control force F1, and control wired 110c is pulled with a second control force F2 different from force F1 (control force F2 is smaller than control force F1, in this example). In this manner, the bending segment 103 can be actuated and bent in a desirable direction (direction of force F1), in accordance with a combination of the driving amounts (strain) or linear displacements of control wires 110a and 110c. To control the posture of the distal end of the steerable instrument 100, driving two (or at least one) of the plurality of control wires 110 would be sufficient. As the forces F1 and F2 are applied to the control wires 110a and 110c, respectively, a corresponding sensor 304 detects the tensile force applied thereto. Here, it should be noted that forces F1 and F2 are not limited to tensile forces exerted by pulling the control wires 110. Forces F1 and F2 can also be compressive forces applied to the control wires 110 by mechanically pushing the control wires by a desired amount of compressive force.

While the case of driving the control wires 110 anchored at the distal end of a single bending section 103 has been described above with respect to FIG. 3B, if control wires of all bending sections of FIG. 3A are driven, the postures of each bending section 103a and 103b can be independently and selectively actuated to bend or twist the steerable section 102 and thereby produce a snake-like movement, depending of the driving amounts of the individual control wires driven by actuators of the actuator system 310 (drive unit). Further, a mechanism that twists or rotates the wire-driven steerable instrument 100 about its longitudinal axis may be additionally provided. In order to provide a certain amount of rotation or twisting action to the steerable instrument 100, a bending section may be first bent in a desirable direction by driving only one control wire 110, and then rotating the entire shaft by actuating a second control wire 110 in a different bending section. Such manipulation of the steerable instrument 100 can be implemented based on well known mechanical and kinematic principles, for example, as described in U.S. Pat. No. 9,144,370 which is hereby incorporated by reference herein for all purposes.

According to one embodiment, the steerable instrument 100 shown in FIG. 3A-3B may have an outer diameter of about 0.14 inches, with a distal steerable section being between 20.0 to 1.0 inches in length, and the total length of the instrument 100 being between 5.0 to 30.0 inches. The anchor members 109 have wire conduits 104 and a tool channel 105 similar to the wire-guide members 108; both are typically constructed from medical-grade plastics or similar composites. These materials allow for fabrication of flexible, yet torsionally resilient steerable instruments, such as catheters and endoscopes of reduced dimensions. For example, other prototype dimensions for a steerable instrument 100 are about 3.3 mm outer diameter (OD), 2.4 mm inner diameter (tool channel), and about 550 mm of total length. These exemplary dimensions are given for the purpose of illustrating sizes and dimensions of steerable instruments disclosed according to the present disclosure. However, a person of ordinary skill in the art will understand that actual dimensions will depend on the specific application of the steerable instrument 100.

Next, robotic and/or manual navigation of the steerable instrument 100 is explained. In general, either during insertion or retraction of the steerable instrument 100 through a patient's anatomy, the center line of the lumen (e.g., an airway) is the desired trajectory to be followed during active control of the bending segments of the steerable section 103. To that end, known guiding techniques of steerable instruments, such as robotic guided catheters or endoscopes, can be used. In general, various known concepts of shaft guidance control the steerable instrument with the goal of forcing the flexible shaft to keep to a desired trajectory. In one such example, when using a shaft guidance system, the steerable instrument is advanced through a lumen while sensors measure the insertion depth of the shaft-guide and the angulations of user-controlled steerable tip segments to obtain trajectory information. The trajectory information is stored in a memory of the system and continuously updated. After a short advance in insertion depth, the shape of the steerable shaft-guide is corrected by adjusting (rotating, twisting, or bending) segments of the instrument in such a way that the new shape closely matches the desired trajectory. This process is repeated until a target area is reached. The same process is applied when the steerable instrument is withdrawn from the patient. See, e.g., US 2007/0135803, which is incorporated by reference herein for all purposes. In some instances, it is also possible to initially manually advance the steerable instrument through a lumen, and then preform robotic control thereafter. In either case, when the steerable instrument 100 travels through a lumen, the bending of the steerable section 103 causes tension and strain on the electrical cable 112.

### Embodiment 1

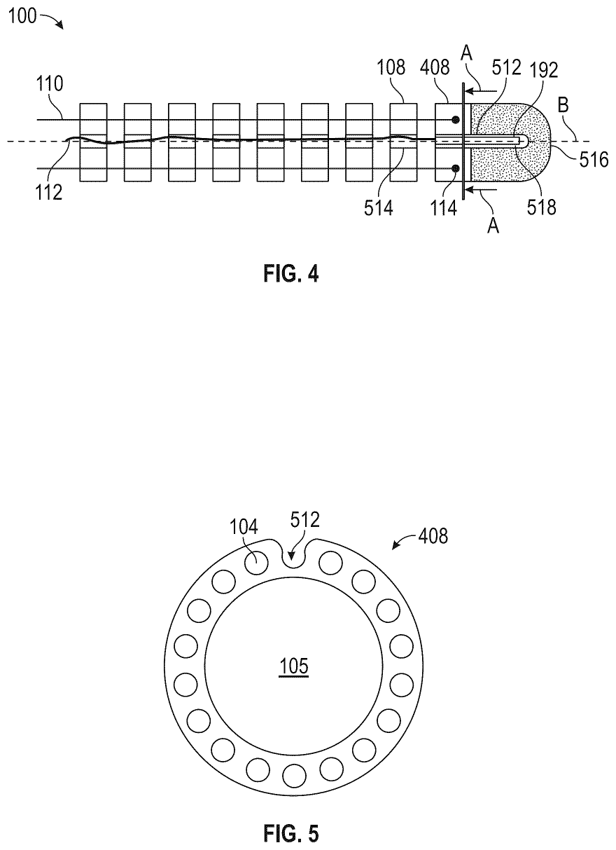

FIG. 4 is a close-up view of a distal part of the steerable instrument 100 in this embodiment. The steerable instrument 100 comprises a distal tip 192, distal guide ring 408, and wire-guide members 108 configured a distance apart from each other about the longitudinal axis B of the steerable instrument 100. The wire-guide members form the bending section to steer distal tip 192 upward, downward and side-to-side in FIG. 4. The distal guide ring 408 has a cross-sectional structure on the A-A line, as seen in FIG. 5. The cross-sectional structure encompasses a first notch 512, a tool channel 105, and wire conduits 104. The tool channel 105 provides access for tools or devices to travel the length of the steerable instrument 100, from the proximal end to distal tip 192. All wire-guide members 108, and the distal guide ring 408 are connected to a backbone (not shown in Figures) running along the longitudinal axis of the steerable instrument 100. The backbone provides bending elasticity and forms the bending section with control wires 110. The distal guide ring 408 includes anchors 114 for securing the control wires 110 to the distal guide ring 408. Control wires 110 go through wire conduits 104 in distal guide ring 408 and wire-guide members 108. Control wires 110 are slide-able in wire conduits 104 along the longitudinal axis B, allowing for the bending.

The opposite end of the control wires 110 are connected to an actuator (see FIG. 2B) and are pushed and pulled to bend the backbone with wire-guide members 108. This bending motion can steer the distal tip 192 in an upward, downward or side directions as seen in FIG. 3B. The distal guide ring 408 also includes a sensor head 516 in first notch 512. The sensor head 516 may be fixed in first notch 512 with an adhesive. Sensor head 516 can be an endoscope camera to provide an endoscopic view or a position/orientation tracking sensor to detect the current position and orientation of distal tip 192. Moreover, distal guide ring 408 is connected to distal tip 192 and forms a rigid distal part with distal tip 192. This rigid distal part is not bendable when the bending section is bent. The sensor head 516 is attached to a groove 518 within distal tip 192, and control wire 110 are overlapped to the direction of the longitudinal axis B and allow the shortening of the total length of the rigid distal part. Since sensor head 516 includes sensor electronics and sensing elements, the shape of the sensor head 516 should be maintained to avoid any deformation to the sensor electronics and the sensing elements inside the sensor head 516. By locating sensor head 516 in first notch 512 and groove 518, the sensor head 516 will be protected from external forces which may cause deformation.

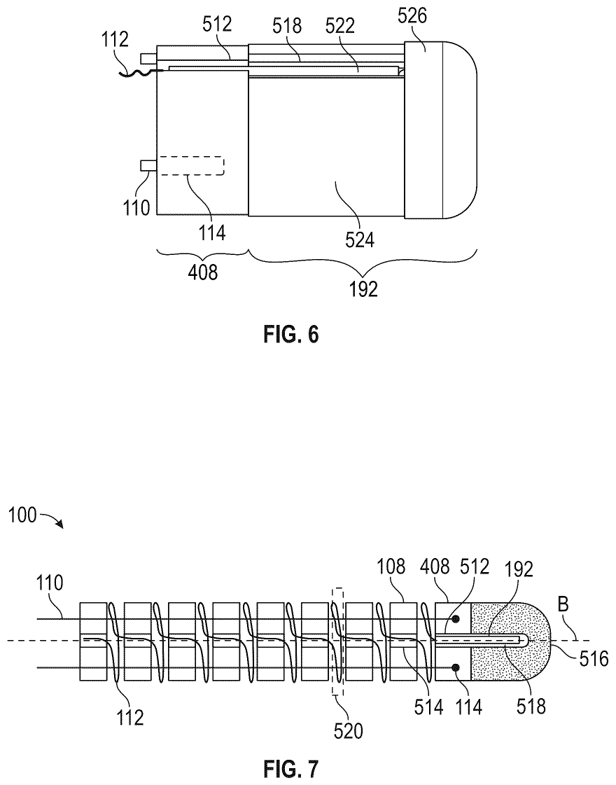

In FIG. 6 we see another example of the design of distal tip 192 and distal guide ring 408 in this embodiment. Here the distal guide ring 408 is made of thermoplastic elastomer, in particular, a polyether block amide. The outer diameter of distal guide ring 408 is approximately 3.8 mm, and the distal guide ring 408 incorporates two control wires 110 both anchored with anchors 114. The driving wires are made of super elastic NiTi alloy with a diameter of approximately 0.15 mm. The anchors 114 are embedded and are attached by the thermal reflow process by leveraging the thermoelastic property of the distal guide ring 408. In turn, this allows the anchors 114 to be set in the distal guide ring 408 during the thermal reflow of the thermoelastic elastomer creating a superior bond that provides the high wire anchor 114 strength, all but eliminating the possibility of the wires slipping or buckling.

The distal guide ring 408 also has a first notch 512, wherein an electromagnetic (“EM”) tracking sensor 522 and sensor head 516 are glued with an adhesive. EM tracking sensor 522 can measure the orientation and position of the distal tip 192 in real-time. This information can be provided to the end user(s) to help operate the steerable instrument 100. The distal tip 192 comprises distal tip body 524 and distal tip cap 526, both may be made of a more rigid material than distal guide ring 408, for example, acrylonitrile butadiene styrene (ABS), nylon or polyether ether ketone (PEEK).

The distal tip body 524 includes a groove 518 and EM tracking sensor 522 in the groove 518. The distal tip cap 526 includes a small projection to engage with the groove 518. With this projection and groove 518, the distal tip cap can be aligned accurately and efficiently to the distal tip body 524. By configuring the distal tip body 524 and distal tip cap 526 in distal tip 192, we can simplify the assembly of distal tip 192. First, the distal guide ring 408 is mounted to an inner tube (not shown in Figure) by mating the tool channel 105 with the inner tube and then glued on the inner tube. Following this step, the distal tip body 524 is also mounted to the inner tube by aligning the groove 518 to first notch 512. Then, the inner tube can be cut to a necessary/desired length at the distal end of distal tip body 524. After this step, the distal tip cap 526 can be attached with the alignment of the small projection with groove 518. Through this assembly, the inner tube length can be determined accurately against the assembly length of distal tip 192 and distal 408 with a simple process.

The EM tracking sensor 522 bridges between the distal tip 192 and the distal guide ring 408 and is glued in both the groove 518 and the first notch 512. Therefore, the EM tracking sensor 522 can strengthen the assembly of the distal tip 192 and the distal guide ring 408. At the same time, because the EM tracking sensor 522 is firmly fixed with the first notch 512 and the groove 518, the position and orientation of the EM tracking sensor 522 against the distal tip 192 is stable and can minimize dislocating the EM tracking sensor 522, providing accurate detection of the position and orientation.

### Embodiment 2

This embodiment includes the same assembly structure of the distal tip 192 and distal guide ring 408 as provided in FIG. 4. In this embodiment, the wire-guide members 108 also embrace the similar cross-sectional structure of the distal guide ring 408, including a tool channel 105 and wire conduits 104, however, this embodiment utilizes a second notch 514. The tool channel 105 delivers access for tools or devices from the proximal end to distal tip 192. The distal tip 192 includes a similar tool channel 105 and allows the tools or devices to exit from distal tip 192. All wire-guide members 108 and the distal guide ring 408 are connected to a backbone (not shown in Figure) running along the longitudinal axis of the steerable instrument 100. The backbone provides bending elasticity and forms the bending section with the control wires 110.

Specifically, the second notches 514 in each of the wire-guide members 108 are aligned along a longitudinal direction, and aligned with the first notch 512, so as to provide a consistent pathway through the steerable instrument 100. Furthermore, the first and second notches 512 and 514, respectively, provide a reference to align all the wire-guide members 108 in the distal guide ring 408 rotationally, for realizing an aligned wire conduit 104 for all control wires 110.

The aligned wire conduits 104 can define the bending orientation accurately and can reduce friction between the control wires 110 and the wire-guide members 108. An electrical cable 112 from the sensor head 516 is located through the aligned second notches 514 to create a path. The electrical cable 112 is slidable within and between the second notches 514 and can adapt to length change in the steerable instrument 100 as it is bent. By aligning the second notches 514, we can guide the electrical cable 112 from the distal to the proximal end of the steerable instrument 100 by avoiding entanglement with the control wires 110. Moreover, since the second notches 514 include openings instead of closed “hole”, we can easily mount the electrical cable 112 from a lateral direction, even when the electrical cable 112 includes a connector or a thicker part on its proximal side.

Furthermore, FIG. 7 provides a schematic of the steerable instrument 100 for another exemplary embodiment. This design example uniquely includes loops 520 of electrical cable 112 between adjacent wire-guide members 108. With this configuration, we can eliminate sliding/rubbing of electrical cable 112 while the loops 520 can accommodate any length change between adjacent second notches 514 in bending without interference with the control wires 110 or tool channel. In addition, the electrical cable 112 may also be fixed on one or more of the second notches 514.

### Embodiment 3

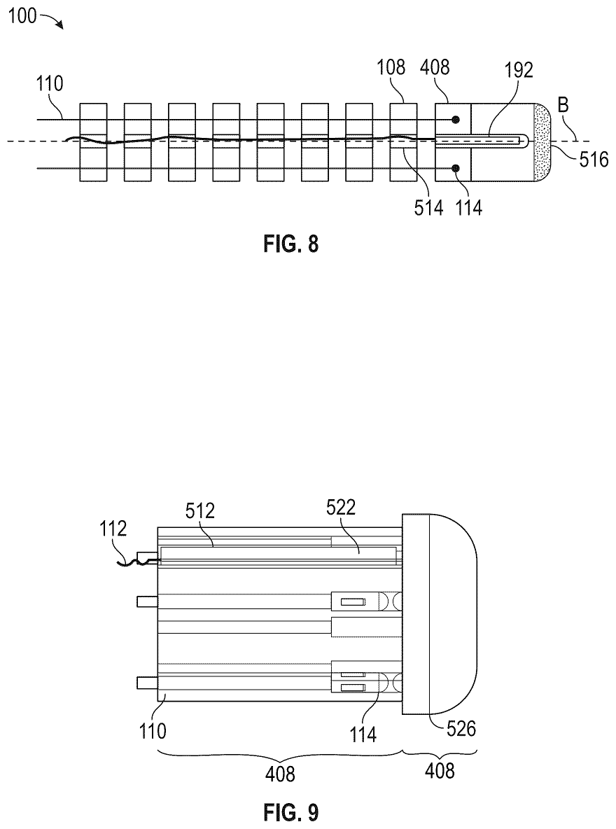

This embodiment includes the same assembly structure as provided in FIG. 4 except for the assembly of distal tip 192 and distal guide ring 408. The distal guide ring 408 in this embodiment includes the sensor head 516 in first notch 512, while distal tip 192 does not include sensor head 516. FIGS. 8 and 9 depict schematics of this embodiment. Instead of the two-piece structure of a distal tip 192 in FIG. 6, the distal tip 192 here includes only the distal tip cap 526. The distal tip cap 526 has projections to mate with distal guide ring 408. By situating the EM tracking sensor 522 only in distal guide ring 408 and configuring the distal tip cap 526 without distal tip body 524, we can still achieve a similar assembly where the inner tube length can be determined accurately against the assembly length of the distal tip 192 and the distal guide ring 408 with a simple process with fewer number of parts.

In referring to the above description, specific details are set forth in order to provide a thorough understanding of the embodiments disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this disclosure belongs. The breadth of the present disclosure is not to be limited by the subject specification, but rather only by the plain meaning of the claim terms employed.

In describing example embodiments illustrated in the drawings, specific terminology is employed for the sake of clarity. However, the disclosure of this patent application specification is not intended to be limited to the specific terminology so selected and it is to be understood that each specific element includes all technical equivalents that operate in a similar manner.

While the present disclosure has been described with reference to exemplary embodiments, it is to be understood that the scope of the following claims is to be accorded the broadest reasonable interpretation so as to encompass all modifications and equivalent structures and functions.

1. A steerable medical instrument, comprising:
a catheter body having a proximal section configured to be mechanically coupled to an actuator, and a distal section configured to be actuated by the actuator, the catheter body having a tool channel which extends along a central axis from a proximal end of the catheter body to a distal end of the catheter body, the catheter body having at least one control wire and at least one electrical cable arranged lengthwise along a wall of the catheter body,
the catheter body further comprising a distal tip configured at the distal end of the distal section of the catheter body, the distal tip configured to house at least a portion of an electrical component,
wherein the distal section includes a plurality of wire-guiding members arranged at a predetermined distance from each other so as to form a plurality of void regions alternately interposed with the plurality of wire-guiding members in the lengthwise direction of the catheter body, and a distal guide ring configured at the distal end of the distal section for attaching the at least one control wire,
wherein the at least one control wire allows the distal guide ring to be in communication with the actuator and at least one control wire is configured to transfer an actuating force from the actuator to the distal guide ring of the catheter body so as to bend at least a portion of the distal section,
wherein the electrical cable is configured to establish an electrical connection between an electrical circuit located outside the catheter body and the electronic component arranged within the distal tip, and
wherein the electrical component bridges the distal tip and distal guide ring.

2. The steerable medical instrument of claim 1, wherein the electrical component is selected from the groups consisting of an endoscope camera, a position sensor, an orientation sensor, or combinations thereof.

3. The steerable medical instrument of claim 1, wherein the distal tip comprises a cap to protect the distal tip.

4. The steerable medical instrument of claim 3, wherein the distal tip comprises a first notch for housing at least a portion of the electrical component.

5. The steerable medical instrument of claim 1, wherein each of the wire-guiding members comprises a second notch for passage of the electrical cable through the distal section.

6. The steerable medical instrument of claim 1, wherein the electrical cable includes strain relief elements arranged in one or more void region such that when the electrical cable is subjected to a tensile load by the actuating force bending the distal section of the catheter body, the strain relief elements within the one or more void regions eliminates the tensile load on the electrical cable.

7. The steerable medical instrument according to claim 6,
wherein the strain relief elements include first portions of the electrical cable which are loosely arranged within the one or more void regions such that, within the one or more void regions, the first portions of the electrical cable can freely move, slide, and/or tighten in response to the actuating force bending the distal section to thereby provide strain relief to, or eliminate the tensile load of, the electrical cable.

8. The steerable medical instrument according to claim 1, further comprising:
an inner sheath configured to enclose at least part of the tool channel,
wherein the one or more wire-guiding members and the void regions interposed with the one or more wire-guiding members are arranged around the inner sheath,
wherein both the electrical cable and the control wires surround the inner sheath,
wherein the one or more wire-guiding members are attached to the inner sheath by reflowing material of the wire-guiding members and/or material of the inner sheath onto each other.

9. The steerable medical instrument according to claim 1, further comprising:
an outer sheath configured to enclose at least part of the distal section of the catheter body,
wherein the outer sheath encloses the one or more wire-guiding members and the void regions interposed with the one or more wire-guiding members,
wherein the outer sheath surrounds both the electrical cable and the control wires within the void regions interposed with the one or more wire-guiding members, and
wherein, when the tensile load by the actuating force bends the distal section of the catheter body, the outer sheath is configured to prevent prolapse of the electrical cable and/or control wires within the void regions interposed with the one or more wire-guiding members.

10. The steerable medical instrument according to claim 1,
wherein the electrical cable is made of one or more strands of electrically conducing wire,
wherein at least part of the one or more strands of electrical conducting wire are wound into a twisted pair cable or weaved into a braided multi-strand cable.
